(19) United States
(12) Patent Application Publication
(10) Pub. No.: US 2026/0314930 A1
(43) Pub. Date: Oct. 15, 2026

(54) HYPERSPECTRAL IMAGING SYSTEM AND METHODS FOR SIMULTANEOUS RETINAL OXYGENATION AND VESSEL MEASUREMENT

(71) Applicant: Board of Regents, The University of Texas System, Austin, TX (US)

(72) Inventors: Baowei Fei, Frisco, TX (US); Minh Tran, Richardson, TX (US)

(21) Appl. No.: 19/461,336

(22) Filed: Jan. 27, 2026

Related U.S. Application Data
(60) Provisional application No. 63/750,249, filed on Jan. 27, 2025.

(51) Int. Cl. A61B 3/12; A61B 3/15; A61B 5/1455; G06T 7/00; G06T 7/11; G06T 7/33; G06T 7/62; G06T 7/80; H04N 23/16; H04N 23/50; H04N 23/55

(52) U.S. Cl. CPC A61B 3/12; A61B 3/15; A61B 5/14551; G06T 7/0012; G06T 7/11; G06T 7/337; G06T 7/62; G06T 7/80; H04N 23/16; H04N 23/55; H04N 23/555; G06T 2207/10068; G06T 2207/20084; G06T 2207/30041; G06T 2207/30101

(57) ABSTRACT

A system and method for imaging a retina, the system and method includes receiving a first image having high spatial resolution and a second image having spectral information, where the first image and the second image are captured simultaneously using a common endoscopic optical probe, generating a high-resolution hyperspectral image by combining spatial information from the first image with spectral information from the second image, and identifying pixels corresponding to retinal blood vessels within the high-resolution hyperspectral image. The system and method may further include determining oxygenation values for the identified blood vessel pixels using the spectral information and determining physical dimensions of the retinal blood vessels using spatial measurements derived from the high-resolution hyperspectral image.

### STATEMENT REGARDING FEDERALLY SPONSORED RESEARCH

This invention was made with Government support under Federal Grant Nos. R01 CA288379 and R01 CA204254 awarded by the National Institutes of Health (NIH) of the U.S. Department of Health and Human Services. The Federal Government has certain rights to this invention.

### BACKGROUND

Retinal imaging and measurement involve capturing images of the retina to visualize anatomical structures and analyze physiological characteristics of retinal tissue. Imaging techniques may include color imaging and multispectral or hyperspectral imaging to obtain spatial and spectral information from the retina. Measured parameters can include retinal blood vessel geometry, such as vessel diameter and branching patterns, as well as biochemical properties derived from spectral responses. These measurements are generally used to generate maps and metrics representing retinal structure and function.

### BRIEF SUMMARY

Simultaneous retinal oxygenation and vessel measurement is described.

In a first aspect of the invention, there is a system for imaging a retina including: an optical assembly comprising an endoscopic optical probe, the optical assembly being configured to simultaneously direct light from the retina to a first imaging sensor and a second imaging sensor, where the first imaging sensor is configured to capture a first image having higher spatial resolution, where the second imaging sensor is configured to capture a second image having spectral information across multiple wavelengths, where simultaneously directing light from the retina to the first imaging sensor and the second imaging sensor comprises directing the light through a beam splitter, and where the beam splitter is configured to divide light from a single optical path into separate optical paths toward the first imaging sensor and the second imaging sensor.

In another aspect of the invention, there is a computer program product including one or more computer readable storage media having program instructions collectively stored on the one or more computer readable storage media. The program instructions are executable to cause a processor to perform operations, including: receiving a first image having high spatial resolution and a second image having spectral information, where the first image and the second image are captured simultaneously using a common endoscopic optical probe; generating a high-resolution hyperspectral image by combining spatial information from the first image with spectral information from the second image; identifying pixels corresponding to retinal blood vessels within the high-resolution hyperspectral image; determining oxygenation values for the identified blood vessel pixels using the spectral information; and determining physical dimensions of the retinal blood vessels using spatial measurements derived from the high-resolution hyperspectral image.

In another aspect of the invention, there is a method for imaging a retina, including: simultaneously directing light from the retina to a first imaging sensor and a second imaging sensor; capturing, by the first imaging sensor, a first image having higher spatial resolution; capturing, by the second imaging sensor, a second image having spectral information; generating a high-resolution hyperspectral image by combining spatial information from the first image with spectral information from the second image; identifying pixels corresponding to retinal blood vessels within the high-resolution hyperspectral image; determining oxygenation values for the identified blood vessel pixels using the spectral information; and determining physical dimensions of the retinal blood vessels using spatial measurements derived from the high-resolution hyperspectral image.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates a representational diagram depicting stages of a known retinal imaging and analysis workflow.

FIG. 2 shows an optical assembly apparatus in accordance with aspects of the present invention.

FIG. 3 shows a flow diagram of an example method in accordance with aspects of the present invention.

FIG. 4A shows a block diagram of an image preprocessing workflow of a super-resolution network architecture in accordance with aspects of the present invention.

FIG. 4B shows a block diagram of the super-resolution network architecture in accordance with aspects of the present invention.

FIG. 5 shows a calibration workflow in accordance with aspects of the present invention.

FIG. 6 shows a schematic diagram of a computing device in accordance with aspects of the present invention.

### DETAILED DESCRIPTION

Simultaneous retinal oxygenation and vessel measurement is described. More particularly, the disclosure relates to dual-camera hyperspectral imaging systems that simultaneously capture high-resolution spatial information and multi-wavelength spectral information from retinal tissue through a common optical path and having the same field of view. The systems and methods described herein may enable enhanced medical diagnostics through automated quantitative analysis of retinal biomarkers, including blood vessel morphology and tissue oxygenation levels. In embodiments, additional clinically relevant measurements may be extracted from the same fused data, including distances between retinal landmarks (e.g., distance from an optic nerve head to a fovea), micro-vasculature network metrics (e.g., micro-vasculature density), and/or reflectance spectra for selected regions of interest (e.g., optic nerve head, macula, and/or deposits within the retina).

Known retinal imaging approaches face several technical limitations that restrict their diagnostic utility. Traditional cameras (e.g., fundus cameras) capture high-resolution color images but provide only three spectral channels corresponding to red, green, and blue (RGB) wavelengths. This limited spectral information constrains the ability to discriminate between different tissue types and prevents accurate quantification of physiological parameters such as oxygen saturation. Hyperspectral imaging systems may overcome this spectral limitation by capturing dozens of wavelength bands across the visible and near-infrared spectrum. However, hyperspectral cameras suffer from inherently lower spatial resolution compared to conventional RGB cameras due to fundamental tradeoffs in sensor design. Sequential acquisition of RGB and hyperspectral images introduces temporal misalignment, particularly problematic when imaging living subjects where eye movements and physiological changes occur between captures. Manual post-acquisition alignment of multi-modal images is time-consuming, operator-dependent, and may introduce registration errors that degrade subsequent analysis. Furthermore, known approaches rely on subjective visual assessment of retinal features rather than objective quantitative measurements, leading to inter-observer variability and reduced diagnostic reproducibility.

Implementations of the invention address these limitations through several technical innovations rooted in computer technology and optical engineering. A dual-camera optical assembly may simultaneously direct light from retinal tissue to both a high-resolution RGB camera and a multi-channel hyperspectral camera through a common optical path. A beam splitter may divide the optical signal, ensuring that both cameras capture identical fields of view at the same instant in time. This simultaneous acquisition eliminates temporal misalignment issues and may obviate the need for complex post-acquisition registration. The system may employ a trained neural network to generate super-resolution hyperspectral images that may combine the high spatial resolution of the RGB camera with the rich spectral information of the hyperspectral camera. This computational fusion produces image data with properties that may not be directly capturable by either camera alone. For example, the super-resolution hyperspectral image may provide vessel diameter measurements at micrometer-scale precision across sixteen or more wavelength channels simultaneously, enabling spatially-resolved oxygen saturation mapping at individual vessel segments that may be smaller than the native resolution of the hyperspectral sensor. In embodiments, automated image processing algorithms may extract quantitative biomarkers including precise vessel diameter measurements and spatially-resolved oxygen saturation maps. These measurements may be performed consistently and reproducibly without subjective operator interpretation.

The technical improvements provided by the disclosed systems may enhance computer functionality in several ways. The super-resolution neural network transforms low-resolution multi-channel data into high-resolution multi-channel data through learned feature extraction and spatial up-sampling operations that may leverage complementary information from the RGB modality. This transformation requires sophisticated tensor operations, convolutional neural network architectures, and/or adversarial training procedures that may be implemented on specialized computing hardware such as graphics processing units. The segmentation and analysis algorithms process high-dimensional hyperspectral image cubes to identify anatomical structures and/or compute physiological parameters through multi-wavelength curve fitting and/or spectral unmixing operations. These computational tasks may involve processing millions of pixels across dozens of spectral channels, requiring substantial memory bandwidth and parallel processing capabilities. The entire processing pipeline from image acquisition through final biomarker extraction may be automated, reducing the computational burden on human operators and enabling consistent, repeatable measurements across large patient populations and/or longitudinal studies.

The methods and systems described herein may not be practicably performed mentally or with pen and paper. The simultaneous capture and precise temporal synchronization of two independent camera systems may require electronic triggering mechanisms and digital control systems. The registration of multi-modal images may involve feature detection algorithms such as SURF or ORB that may process thousands of image features to compute optimal geometric transformations. The super-resolution neural network may contain millions of trainable parameters and may require thousands of gradient descent iterations on large training datasets, operations that may be computationally infeasible without modern computing hardware. The spectral unmixing and oxygenation mapping may involve solving non-linear optimization problems at each pixel across 16 or more wavelength channels, requiring numerical methods and/or iterative solvers. The vessel segmentation may employ deep learning architectures that may analyze spatial and spectral patterns across the entire image simultaneously. These operations may collectively represent a technological solution to the technical problem of obtaining high-resolution, multi-spectral retinal images with quantitative biomarker extraction.

The disclosed approaches may be applicable to various computer-implemented imaging and analysis solutions beyond the specific retinal imaging context. The general principle of fusing high-resolution spatial data with low-resolution spectral data through neural network processing may be applied to other medical imaging modalities, remote sensing applications, industrial inspection systems, and/or any domain where simultaneous capture of complementary image modalities may be beneficial. The automated biomarker extraction methods may be adapted to analyze different anatomical structures and/or tissue types. The calibration and measurement conversion techniques may be generalized to other optical imaging systems where physical dimensions may need to be derived from digital image data. The adversarial training approach with separate spatial and spectral discriminators may be employed in other super-resolution or image fusion applications where multiple quality metrics may need to be optimized simultaneously.

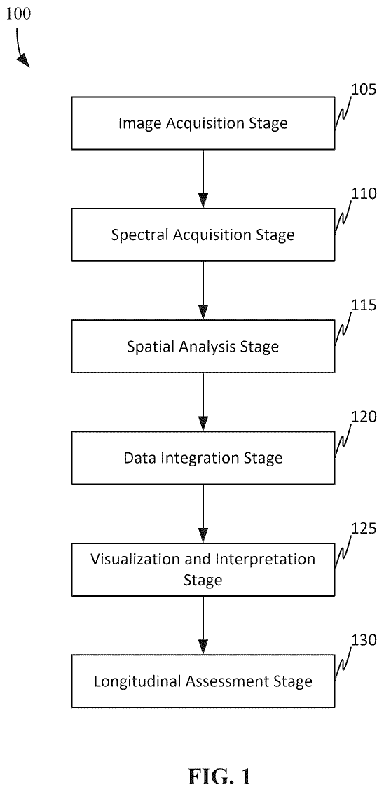

FIG. 1 illustrates a representational diagram depicting stages of a known retinal imaging and analysis workflow. Specifically, FIG. 1 provides an overview of known retinal imaging workflows used to assess retinal vasculature and blood oxygenation and highlights their limitations. Referring now to FIG. 1, in image acquisition stage 105, a retinal image is captured using either a standard fundus camera or a hyperspectral imaging device. However, standard fundus cameras provide only RGB spatial information without spectral content, while hyperspectral imaging devices typically acquire images at low spatial resolution or require sequential scanning. As a result, oxygenation information and fine microvascular detail cannot be reliably obtained within a single image capture.

At spectral acquisition stage 110, hyperspectral data is collected to enable estimation of retinal oxygenation based on wavelength-dependent absorption characteristics of hemoglobin. This process is limited by hardware constraints, including long acquisition times or reduced spatial resolution when snapshot hyperspectral cameras are used. For example, snapshot hyperspectral sensors allocate detector pixels across multiple wavelength bands, resulting in a substantially reduced effective spatial resolution. Thus, small retinal vessels and localized oxygenation variations may not be accurately resolved.

At spatial analysis stage 115, retinal vessel diameters and anatomical features are measured using high-resolution RGB images or separate imaging modalities. This process is limited because spatial measurements are typically performed independently from spectral analysis and often rely on manual or semi-automatic measurement techniques. For example, vessel diameters may be measured from images acquired at a different time or using a different imaging device than those used for oxygenation analysis. Thus, spatial and spectral data are not inherently co-registered, leading to alignment errors and inconsistent measurements.

At data integration stage 120, outputs from spatial analysis and spectral analysis are combined to infer retinal health metrics, such as vessel caliber and oxygen saturation. This process is limited because integration is performed after image acquisition using separately processed datasets. For example, eye motion, focus drift, or illumination variation between image captures may introduce inconsistencies. Thus, known workflows are unable to reliably produce simultaneous, pixel-level measurements of retinal oxygenation and vessel diameter from a single acquisition.

At visualization and interpretation stage 125, combined retinal metrics are displayed for clinical review or research analysis. This process is limited because the underlying measurements are derived from temporally or spatially misaligned data. For example, apparent changes in vessel diameter or oxygenation may reflect imaging artifacts rather than physiological changes. Thus, known workflows may produce results that are difficult to interpret, reproduce, or use for consistent assessment.

At longitudinal assessment stage 130, retinal measurements obtained from multiple imaging sessions are compared to evaluate disease progression or treatment response. This process is limited in known workflows because variations in image acquisition, spatial alignment, and measurement accuracy between sessions reduce repeatability. For example, differences in vessel segmentation or oxygenation estimation across visits may obscure true physiological changes. Thus, known workflows are poorly suited for reliable longitudinal monitoring or quantitative trend analysis.

Implementations of the present invention improve upon the foregoing method by providing simultaneous acquisition of high-resolution spatial data and multi-wavelength spectral data through a dual-camera optical assembly with a common optical path and identical field of view, thereby eliminating temporal and spatial misalignment. The system may employ neural network-based super-resolution processing to generate high-resolution hyperspectral images that may combine spatial detail from the RGB camera with spectral fidelity from the hyperspectral camera. Automated segmentation and quantitative analysis algorithms may extract vessel diameter measurements and oxygen saturation values directly from the fused image data without manual intervention. The simultaneous capture and computational fusion may enable pixel-level co-registration of spatial and spectral information, providing consistent, reproducible measurements suitable for longitudinal monitoring and quantitative trend analysis.

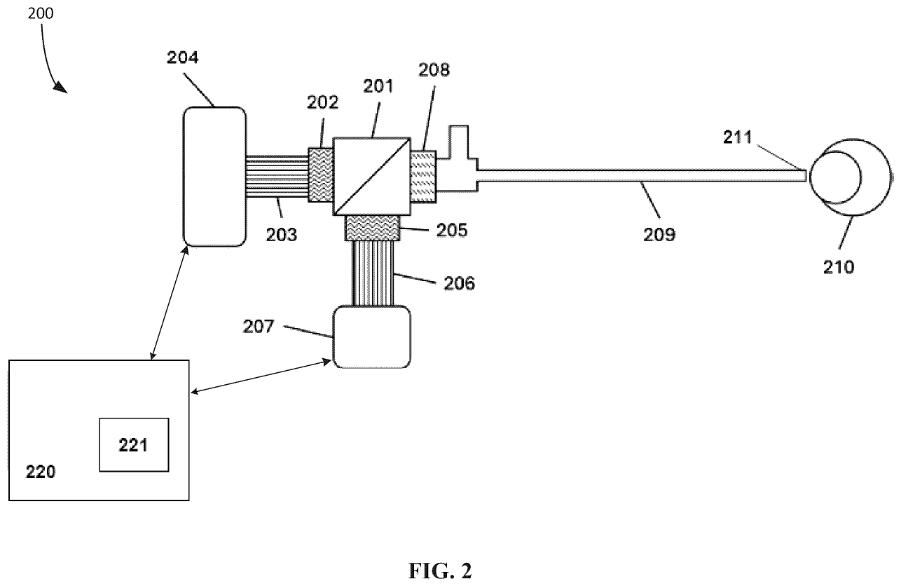

FIG. 2 shows an optical assembly apparatus 200 in accordance with aspects of the present invention. Referring to FIG. 2, optical assembly apparatus 200 may be configured to enable simultaneous capture of high spatial resolution RGB images and spectral information across multiple wavelengths from a single optical path. The optical assembly 200 may comprise an endoscopic optical probe configuration that may direct light reflected from retinal tissue to both a first imaging sensor 204 and a second imaging sensor 207 concurrently. The beam splitter 201 may be positioned within the optical path to divide incoming light into separate optical paths toward the first imaging sensor 204 and the second imaging sensor 207. This configuration may overcome temporal and spatial misalignment issues present in known workflows by ensuring that both imaging sensors capture data from the same field of view at the same instant in time.

In embodiments, the optical assembly 200 may include endoscope 209 (e.g., a telescopic endoscope) that may provide an optical pathway from cornea surface 210 to the beam splitter 201. The endoscope 209 may have a proximal end 211 that may be positioned near or in contact with the cornea surface 210 during image acquisition. In embodiments, the endoscope 209 may be configured with an outer diameter appropriate for the subject being imaged. For example, in some embodiments, the endoscope 209 may have an outer diameter of approximately 3 millimeters for mouse retinal imaging. In other embodiments, the endoscope 209 may have an outer diameter of approximately 7 millimeters for human retinal imaging. In embodiments, the endoscope 209 may incorporate integrated fiber optic illumination to provide light to the retinal surface.

In embodiments, the beam splitter 201 may be attached to the endoscope 209 via a thread adapter 208. The beam splitter 201 may be configured to divide light received from the endoscope 209 into two separate optical paths. In some embodiments, the beam splitter 201 may divide light in a 50:50 ratio. In other embodiments, the beam splitter 201 may divide light in a 70:30 ratio. In further embodiments, the beam splitter 201 may divide light in a 90:10 ratio. Here, a 70:30 ratio means that 70% of the incoming light gets transmitted into the high-resolution imaging camera, and 30% of the light gets reflected by the beamsplitter into the hyperspectral imaging camera. A 90:10 ratio means that 90% of the incoming light gets transmitted into the high-resolution imaging camera, and 10% of the light gets reflected by the beamsplitter into the hyperspectral imaging camera. The specific division ratio may be selected based on the relative light sensitivity requirements of the first imaging sensor 204 and the second imaging sensor 207.

In embodiments, the first imaging sensor 204 may be configured to capture high spatial resolution RGB images. The first imaging sensor 204 may comprise a scientific grade RGB camera with high pixel density. In some embodiments, the first imaging sensor 204 may have a resolution ranging from 2048×3072 pixels to 3840×3040 pixels. The first imaging sensor 204 may be configured to capture three-channel color imaging corresponding to red, green, and blue (RGB) wavelengths. In embodiments, the first imaging sensor 204 may be connected to the beam splitter 201 via a first optical path that may include a first lens 203 and a first thread adapter 202. The first lens 203 may have a fixed focal length. In some embodiments, the first lens 203 may have a focal length of approximately 50 millimeters. The first thread adapter 202 may mechanically and optically couple the first lens 203 to the beam splitter 201. Of course, any suitable mechanical and/or physical coupler may be used in place of first thread adaptor 202 and/or second thread adaptor 205.

In embodiments, the second imaging sensor 207 may be configured to capture spectral information across multiple wavelengths. The second imaging sensor 207 may comprise a snapshot hyperspectral imaging camera. The second imaging sensor 207 may utilize a mosaic layout incorporating a Fabry-Perot interferometer to separate incoming light into multiple spectral bands. In some embodiments, the second imaging sensor 207 may capture 16 spectral bands. The second imaging sensor 207 may have a lower spatial resolution than the first imaging sensor 204. In some embodiments, the second imaging sensor 207 may have a resolution of approximately 270×512 pixels. The second imaging sensor 207 may be configured to capture spectral information across a wavelength range. In some embodiments, the wavelength range may be approximately 460 nanometers to 600 nanometers. In other embodiments, the wavelength range may be approximately 600 nanometers to 800 nanometers. In embodiments, the second imaging sensor 207 may be connected to the beam splitter 201 via a second optical path that may include a second lens 206 and a second thread adapter 205. The second lens 206 may have a fixed focal length. In some embodiments, the second lens 206 may have a focal length of approximately 75 millimeters. The second thread adapter 205 may mechanically and optically couple the second lens 206 to the beam splitter 201.

In embodiments, the optical assembly 200 may be operatively connected to a control computer 220. The control computer 220 may contain a software program 221. The control computer 220 may be configured to send trigger signals to both the first imaging sensor 204 and the second imaging sensor 207 simultaneously. The simultaneous triggering may ensure that both imaging sensors capture data at the same instant in time. The control computer 220 may receive image data from both the first imaging sensor 204 and the second imaging sensor 207. The control computer 220 may be configured to store the received image data for subsequent processing. In embodiments, prior to capturing retinal images, the control computer 220 may cause acquisition of calibration images comprising white reference images of a high-reflectance target (e.g., a Spectralon tile) positioned at substantially the same working distance as the imaged eye (e.g., approximately an eye diameter away from a distal tip of the endoscopic optical probe), thereby enabling radiometric correction of one or both modalities. In embodiments, the trigger signal may be generated responsive to manual operator input, and in other embodiments the control computer 220 may automatically detect an optimal focus condition and automatically generate the trigger signal. In some cases, control computer 220 may include instructions and processing capabilities for image processing of the captured images. In some cases, control computer 220 may communicate with a separate device for image processing operations.

In embodiments, the first imaging sensor 204 may operate with an exposure time. In some embodiments, the exposure time for the first imaging sensor 204 may range from approximately 70 milliseconds to approximately 100 milliseconds. The second imaging sensor 207 may operate with an exposure time. In some embodiments, the exposure time for the second imaging sensor 207 may range from approximately 150 milliseconds to approximately 700 milliseconds. The exposure times may be selected to ensure adequate signal-to-noise ratio while maintaining safe illumination levels for retinal tissue.

In embodiments, the optical assembly 200 may be configured to maintain illumination power within safe limits for retinal exposure. In some embodiments, the illumination power may be maintained at or below approximately 180 microwatts. The illumination may be provided by a light source. In some embodiments, the light source may comprise a Xenon lamp. In other embodiments, the light source may comprise a Halogen lamp. The light from the light source may be delivered to the retinal surface via a fiber optic light guide that may travel parallel to the endoscope 209.

The optical assembly 200 may be mounted on positioning stages to enable precise alignment with the subject's eye. The positioning stages may provide multiple degrees of freedom for adjustment. In some embodiments, the positioning stages may provide four degrees of freedom comprising three rotational adjustments and one translational adjustment. A horizontal translation stage may enable forward and backward movement of the optical assembly 200. An articulation or rotation stage may enable angular adjustment of the optical assembly 200. A vertical translation platform may enable positioning of the subject relative to the optical assembly 200.

In embodiments, the beam splitter 201 may be configured to preserve spectral content across both output paths. The beam splitter 201 may maintain identical field of view for both the first imaging sensor 204 and the second imaging sensor 207. The beam splitter 201 may have transmission characteristics optimized for the wavelength ranges captured by both imaging sensors. The beam splitter 201 may be positioned such that the optical path length from the beam splitter 201 to the first imaging sensor 204 may be substantially equal to the optical path length from the beam splitter 201 to the second imaging sensor 207.

In embodiments, the first lens 203 and the second lens 206 may be configured to focus light onto the respective imaging sensors. The focal lengths of the first lens 203 and the second lens 206 may be selected to achieve desired magnification factors. The magnification factors may determine the relationship between physical dimensions in the retinal tissue and pixel dimensions in the captured images. The first lens 203 may be adjustable to optimize focus for the first imaging sensor 204. The second lens 206 may be adjustable to optimize focus for the second imaging sensor 207.

In embodiments, the optical assembly 200 may enable pixel-level co-registration of spatial and spectral data by ensuring that both imaging sensors view the same retinal region simultaneously through a common optical path. The beam splitter 201 may divide the light such that spatial features visible in the high-resolution RGB image captured by the first imaging sensor 204 may correspond to the same spatial features in the lower-resolution hyperspectral image captured by the second imaging sensor 207. This correspondence may be maintained because both imaging sensors receive light that has traveled through the same endoscope (e.g., endoscope 209) and beam splitter 201 at the same instant in time.

In embodiments, the configuration of the optical assembly 200 may eliminate the need for sequential image acquisition that may be present in known workflows. Sequential acquisition may introduce temporal misalignment due to eye motion, focus drift, or illumination variation between captures. The simultaneous capture enabled by the optical assembly 200 may ensure that the high spatial resolution RGB image and the spectral information are inherently temporally aligned. The common optical path through the endoscope 209 and beam splitter 201 may ensure that the images are inherently spatially aligned to the same field of view.

In embodiments, the optical assembly 200 may provide a foundation for a dual-sensor imaging approach. The dual-sensor imaging approach may enable subsequent processing to combine the high spatial resolution from the first imaging sensor 204 with the rich spectral information from the second imaging sensor 207. The simultaneous capture and common optical path may enable computational methods to generate high-resolution hyperspectral images that may preserve both spatial detail and spectral fidelity. The pixel-level co-registration enabled by the optical assembly 200 may allow for accurate fusion of spatial and spectral data without the alignment errors that may be present in known workflows that acquire spatial and spectral data separately.

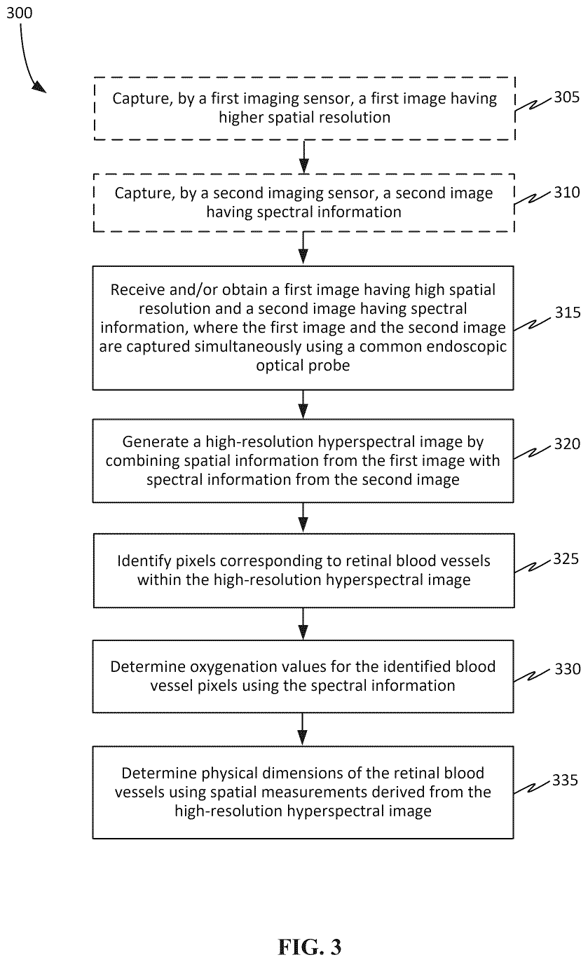

FIG. 3 shows a flow diagram of an example method 300 in accordance with aspects of the present invention. Operations of method 300 are described with reference to elements and actions depicted in and described with reference to FIG. 2.

At operation 305, the system (e.g., first imaging sensor 204 of FIG. 2) may be optionally configured, as indicated by the dashed line, to capture a first image having higher spatial resolution. As noted above, in embodiments, the first imaging sensor may comprise a scientific grade RGB camera with high pixel density configured to capture three-channel color imaging corresponding to red, green, and blue wavelengths. For example, the first imaging sensor may have a resolution ranging from 2048×3072 pixels to 3840×3040 pixels and may operate with an exposure time ranging from approximately 70 milliseconds to approximately 100 milliseconds. In embodiments, operation 305 may be performed in accordance with the descriptions and embodiments of first imaging sensor 204 of FIG. 2.

At operation 310, the system (e.g., second imaging sensor 207 of FIG. 2) may be optionally configured to capture a second image having spectral information. As noted above, in embodiments, the second imaging sensor may comprise a snapshot hyperspectral imaging camera that may utilize a mosaic layout incorporating a Fabry-Perot interferometer to separate incoming light into multiple spectral bands. For example, the second imaging sensor may capture 16 spectral bands across a wavelength range of approximately 460 nanometers to 600 nanometers or approximately 600 nanometers to 800 nanometers, with a resolution of approximately 270×512 pixels and an exposure time ranging from approximately 150 milliseconds to approximately 700 milliseconds. In embodiments, operation 310 may be performed in accordance with the descriptions and embodiments of second imaging sensor 207 of FIG. 2.

At operation 315, the system (e.g., control computer 220 of FIG. 2) is configured to receive and/or obtain a first image having high spatial resolution and a second image having spectral information, where the first image and the second image are captured simultaneously using a common endoscopic optical probe. In embodiments, with reference to features of FIG. 2, the control computer 220 may send synchronized trigger signals to both the first imaging sensor 204 and the second imaging sensor 207 simultaneously to ensure that both imaging sensors capture data at the same instant in time. The control computer 220 may receive the high-resolution RGB image data from the first imaging sensor 204 and the multi-channel hyperspectral image data from the second imaging sensor 207. In embodiments, the control computer 220 may store the received image data in memory for subsequent processing. The simultaneous triggering may ensure temporal alignment of the image pair, and the common optical path through the endoscope 209 and beam splitter 201 may ensure that both images capture identical fields of view, thereby enabling pixel-level co-registration of spatial and spectral data. In embodiments, operation 315 may be performed in accordance with the descriptions and embodiments of control computer 220 of FIG. 2.

At operation 320, the system (e.g., control computer 220 of FIG. 2 or a computer/system in communication with control computer 220) is configured to generate a high-resolution hyperspectral image by combining spatial information from the first image with spectral information from the second image. In embodiments, with reference to features of FIG. 2, the control computer 220 (or a computer/system in communication with control computer 220) may execute a trained neural network comprising a generator network that may receive the aligned high-resolution RGB image and the aligned low-resolution hyperspectral image as inputs. The generator network may comprise a U-Net architecture with encoder-decoder layers and/or skip connections that may extract spatial features from the RGB image and/or spectral features from the hyperspectral image. The generator network may fuse these features in intermediate representations and may up-sample the hyperspectral data guided by the RGB spatial information to produce a high-resolution hyperspectral image. As used herein, up-sampling refers to increasing the spatial resolution of the hyperspectral data through learned interpolation operations that may predict high-frequency spatial details based on the spatial structure present in the RGB image while preserving the spectral characteristics of the hyperspectral data.

In embodiments, the neural network may employ dual loss functions comprising a spatial loss function that may evaluate spatial fidelity by comparing the generated image with the RGB image using perceptual loss metrics, and/or a spectral loss function that may evaluate spectral accuracy by comparing down-sampled generated data with the original hyperspectral image using root mean square error. In embodiments, the neural network may further employ adversarial training with separate spatial and spectral discriminators that may provide feedback to optimize both spatial detail preservation and spectral fidelity simultaneously. The resulting high-resolution hyperspectral image may combine the high spatial resolution of the RGB camera with the rich spectral information of the hyperspectral camera, producing image data with properties that may not be directly capturable by either camera alone.

At operation 325, the system (e.g., control computer 220 of FIG. 2 or a computer/system in communication with control computer 220) is configured to identify pixels corresponding to retinal blood vessels within the high-resolution hyperspectral image. In embodiments, the control computer 220 (or a computer/system in communication with control computer 220) may execute a vessel segmentation algorithm that may process the high-resolution hyperspectral image to distinguish blood vessel pixels from background tissue pixels. In embodiments, the segmentation algorithm may comprise a trained neural network that may analyze both spatial patterns characteristic of vessel morphology and/or spectral signatures characteristic of blood absorption across the multiple wavelength channels. In embodiments, the segmentation algorithm may identify vessel boundaries with precision enabled by the high spatial resolution of the generated image. In embodiments, the control computer 220 (or a computer/system in communication with control computer 220) may generate a vessel segmentation map that may delineate the precise locations and extents of retinal blood vessels, including arterioles and/or venules. The segmentation map may serve as input for subsequent quantitative measurements of vessel dimensions and/or may provide pixel locations for oxygenation analysis. In some embodiments, the same or an additional segmentation network may additionally identify pixels corresponding to other clinically relevant retinal structures (e.g., macula, optic nerve head, and/or deposits) and generate additional segmentation masks for those structures.

At operation 330, the system (e.g., control computer 220 of FIG. 2 or a computer/system in communication with control computer 220) is configured to determine oxygenation values for the identified blood vessel pixels using the spectral information. In embodiments the control computer 220 (or a computer/system in communication with control computer 220) may apply spectral unmixing algorithms to the multi-channel spectral data at each vessel pixel location identified in the segmentation map. The control computer 220 (or a computer/system in communication with control computer 220) may convert the spectral reflectance data to optical density values and may perform multi-wavelength curve fitting to a hemoglobin absorption model that may account for both oxygenated hemoglobin and deoxygenated hemoglobin contributions. For example, a white-reference corrected intensity value I(λ) may be converted to optical density according to OD(λ)=−log 10(I(λ)). The control computer 220 (or a computer/system in communication with control computer 220) may solve, optionally under non-negative constraints, a multi-wavelength model such as:

OD(λ)=B−N ln(λ)+A[μ_{HbO}_{₂}(λ)+(1−SO₂)μ_{Hb}]

where B and N model scattering contributions, A scales absorbance (e.g., vessel thickness), SO2 is an oxygen saturation parameter, and μ_{HbO}_{₂}(λ) and μ_{Hb}(λ) are wavelength-dependent absorption coefficients for oxygenated and deoxygenated hemoglobin, respectively. The curve fitting may solve for oxygen saturation as a parameter representing the percentage of oxygen-bound hemoglobin at each pixel location. In embodiments, the control computer 220 (or a computer/system in communication with control computer 220) may apply a non-linear optimization algorithm with non-negative constraints to extract oxygen saturation values across 16 or more wavelength channels simultaneously. The control computer 220 (or a computer/system in communication with control computer 220) may generate an oxygenation map that may provide spatially-resolved oxygen saturation measurements at individual vessel segments. The control computer 220 (or a computer/system in communication with control computer 220) may distinguish arterioles from venules based on oxygenation levels, with arterioles typically exhibiting oxygen saturation values above 90 percent and venules typically exhibiting values below 90 percent. In embodiments, a validation procedure may include imaging blood samples with controlled deoxygenation using sodium dithionite concentrations correlated with oxygen saturation, thereby validating oxygenation estimates against known conditions.

At operation 335, the system (e.g., control computer 220 of FIG. 2 or a computer/system in communication with control computer 220) is configured to determine physical dimensions of the retinal blood vessels using spatial measurements derived from the high-resolution hyperspectral image. In embodiments, the control computer 220 (or a computer/system in communication with control computer 220) may apply measurement conversion algorithms that may convert pixel-based measurements to physical dimensions in micrometers. The control computer 220 (or a computer/system in communication with control computer 220) may utilize a calibrated magnification factor that may relate pixel dimensions to physical dimensions based on the optical characteristics of the endoscope 209 and lenses 203, 206. The control computer 220 (or a computer/system in communication with control computer 220) may identify vessel boundaries in the segmentation map and/or may measure the distance between opposing vessel edges in pixel units. In embodiments, the control computer 220 (or a computer/system in communication with control computer 220) may multiply the pixel measurements by the conversion factor to obtain vessel diameters in micrometers. In embodiments, the control computer 220 (or a computer/system in communication with control computer 220) may measure additional vessel characteristics including vessel lengths, tortuosity, density, and/or branching patterns. In embodiments, the high spatial resolution of the generated hyperspectral image may enable vessel diameter measurements at micrometer-scale precision, including measurements of small vessels that may be below the native resolution of the hyperspectral sensor. In embodiments, the control computer 220 (or a computer/system in communication with control computer 220) may generate quantitative biomarker data comprising vessel diameters and morphological characteristics that may be used for disease detection, progression monitoring, and/or treatment response assessment.

Thus, method 300 provides simultaneous acquisition and automated analysis of retinal spatial and/or spectral information for quantitative biomarker extraction. In this manner, method 300 leverages dual-camera simultaneous imaging with neural network-based super-resolution processing to achieve high-resolution hyperspectral imaging that may combine spatial detail with spectral fidelity. Method 300 may eliminate temporal and spatial misalignment issues present in sequential imaging workflows by ensuring both cameras capture identical fields of view at the same instant through a common optical path. Method 300 may enable automated, objective, and reproducible measurements of vessel diameters and oxygen saturation levels without manual intervention or subjective operator interpretation. Method 300 may provide pixel-level co-registration of structural and functional information, enabling spatially-resolved oxygenation mapping at individual vessel segments. Method 300 may produce quantitative biomarkers suitable for longitudinal monitoring, disease progression tracking, and treatment response assessment in both research and clinical applications.

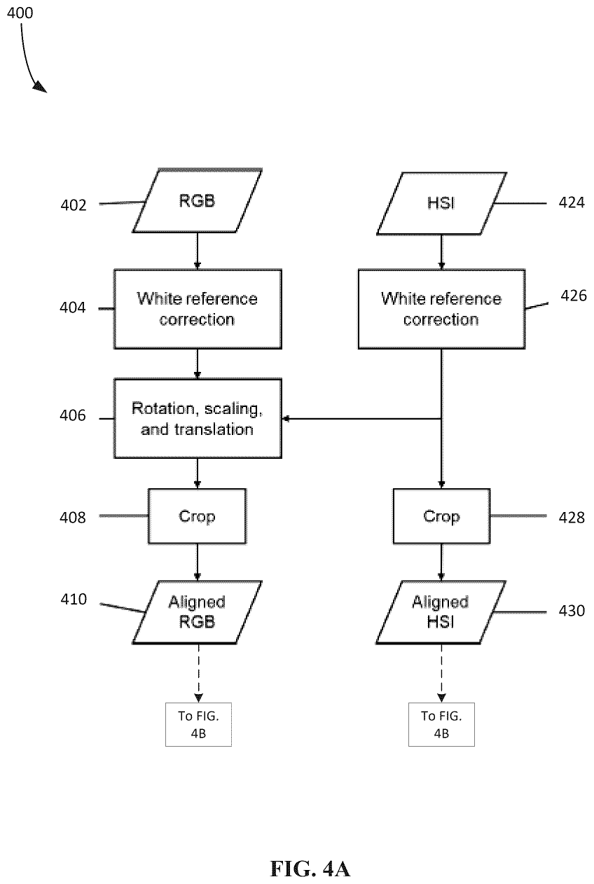

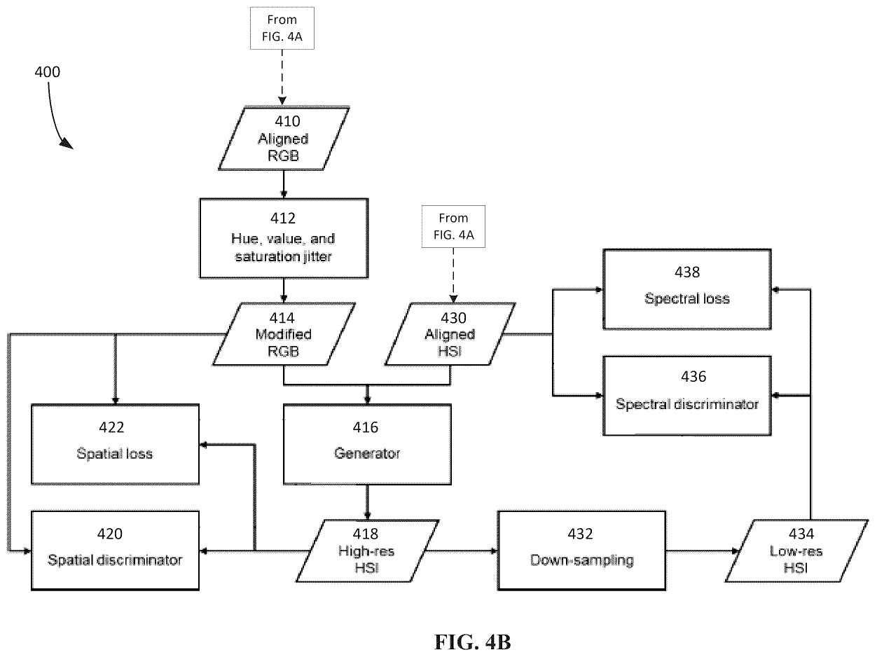

FIG. 4A shows a block diagram of an image preprocessing workflow of a super-resolution network architecture 400 in accordance with aspects of the present invention. The preprocessing workflow of the super-resolution network architecture 400 may prepare RGB and hyperspectral images for super-resolution processing by performing calibration, alignment, and cropping operations. The preprocessing workflow of the super-resolution network architecture 400 may receive an RGB image 402 and a hyperspectral image (HSI) 424 as inputs from a first imaging sensor (e.g., first imaging sensor 204 of FIG. 2) and a second imaging sensor (e.g., second imaging sensor 207 of FIG. 2), respectively.

The RGB image 402 may undergo white reference correction 404 to normalize illumination variations and correct for spectral characteristics of the light source. The white reference correction 404 may divide pixel values by reference measurements obtained from a white calibration target to produce corrected RGB data. For example, the correction may apply the formula I_{corrected}=I_{raw}/I_{white_reference}, where each raw pixel value may be divided by the corresponding white reference pixel value captured under identical illumination conditions.

In embodiments, the corrected RGB data may then undergo rotation, scaling, and/or translation operations 406 to geometrically align the RGB image with the hyperspectral image. In embodiments, the rotation, scaling, and/or translation operations 406 may apply a transformation matrix computed from feature correspondences between the RGB and hyperspectral images. For example, the transformation may employ Speeded-Up Robust Features (SURF) and/or Oriented FAST and Rotated BRIEF (ORB) feature detection algorithms to identify matching features between the two images, followed by computation of a similarity transformation matrix that may map RGB pixel coordinates to hyperspectral pixel coordinates.

In embodiments, the aligned RGB data may then undergo a crop operation 408 to extract a region of interest corresponding to the field of view captured by one or both sensors. The crop operation 408 may produce an aligned RGB image 410 having spatial dimensions and field of view matched to the hyperspectral image. For example, the crop operation may extract a center region from the transformed RGB image, thereby removing edge artifacts introduced by the geometric transformation and ensuring that the aligned RGB image 410 may have dimensions that may be an integer multiple of the hyperspectral image dimensions.

In embodiments, the HSI 424 may undergo white reference correction 426 using the same white calibration target measurements to normalize illumination and correct spectral response. For example, the correction may apply the formula.

$${I}_{corrected} = \frac{{I}_{original}}{{I}_{white}}$$

where I_{corrected} refers to the image that has been white reference corrected. I_{original} refers to the original image prior to correction. I_{white} refers to a white reference image captured prior. In such embodiments, I_{white} should have the same dimension and number of channels as I_{original}.

In embodiments, the corrected hyperspectral data may then undergo a crop operation 428 to extract the same region of interest as the RGB image. The crop operation 428 may produce an aligned HSI image 430 having the same field of view as the aligned RGB image 410. For example, the crop operation may extract a center region that may correspond spatially to the cropped RGB region, thereby ensuring pixel-level correspondence between the two modalities for subsequent super-resolution processing. In embodiments, the aligned RGB image 410 and/or the aligned HSI image 430 may be provided as inputs to the super-resolution network architecture illustrated in FIG. 4B.

FIG. 4B shows a block diagram of the super-resolution network architecture 400 in accordance with aspects of the present invention. In embodiments, the super-resolution network architecture 400 may receive the aligned RGB image 410 and the aligned HSI image 430 from the preprocessing workflow of FIG. 4A. The aligned RGB image 410 may undergo hue, value, and saturation jitter operations 412 that may randomly adjust color parameters by small amounts to introduce spectral variation during training. In embodiments, the jitter operation may produce a modified RGB image 414 that may retain spatial information while providing data augmentation to improve neural network generalization. For example, the jitter operation may randomly adjust hue by ±10 degrees, saturation by ±15 percent, and/or value by ±15 percent to create spectral diversity in the training data while maintaining spatial structure.

The modified RGB image 414 and the aligned HSI image 430 may be input into a generator network 416. As noted above, a generator network 416 may comprise a U-Net architecture with encoder-decoder layers and skip connections. The generator network 416 may extract spatial features from the modified RGB image 414 and spectral features from the aligned HSI image 430, fuse these features in intermediate representations, and/or up-sample the hyperspectral data guided by the RGB spatial information. For example, the encoder pathway may progressively down-sample both inputs through convolutional layers to extract multi-scale features, the decoder pathway may progressively up-sample the fused features through transposed convolutional layers, and/or skip connections may concatenate encoder features with decoder features at corresponding spatial scales to preserve fine spatial details during up-sampling. As used herein, down-sampling refers to reducing the spatial resolution of image data through pooling and/or strided convolution operations that may aggregate neighboring pixel and/or removing pixels values to produce a smaller representation while preserving essential features at coarser spatial scales.

In embodiments, the generator network 416 may produce a high-resolution HSI image 418 having the same spatial dimensions as the modified RGB image 414 and the same number of spectral channels as the aligned HSI image 430. For example, if the modified RGB image 414 may have dimensions of 2048×3072 pixels and the aligned HSI image 430 may have 16 spectral channels, the high-resolution HSI image 418 may have dimensions of 2048×3072×16, combining high spatial resolution with rich spectral information.

In embodiments, the high-resolution HSI image 418 may undergo a down-sampling operation 432 using a bi-linear sampling algorithm. In such embodiments, the down-sampling operation 432 may reduce the spatial dimensions to match those of the aligned HSI image 430. For example, the down-sampling operation may reduce the 2048×3072 spatial dimensions by a factor of four in each dimension to produce 512×768 dimensions. In embodiments, the down-sampling operation 432 may produce a low-resolution HSI image 434 having the same spatial dimensions and/or number of spectral channels as the aligned HSI image 430. For example, the low-resolution HSI image 434 may have dimensions of 512×270×16 pixels, matching the original hyperspectral camera output for spectral fidelity comparison.

In embodiments, a spectral discriminator 436 may receive the low-resolution HSI image 434 and/or the aligned HSI image 430 as inputs and may evaluate whether the spectral characteristics of the low-resolution HSI image 434 match those of the aligned HSI image 430. For example, the spectral discriminator 436 may employ convolutional layers that may analyze spectral signatures across the 16 wavelength channels to determine whether the down-sampled generated data may exhibit spectral characteristics consistent with real hyperspectral measurements, thereby providing adversarial feedback to encourage spectral accuracy.

In embodiments, a spectral loss function 438 may compare the low-resolution HSI image 434 with the aligned HSI image 430 to evaluate spectral fidelity. In embodiments, the spectral loss function 438 may comprise a root mean square error metric. For example, the spectral loss may compute the square root of the mean squared difference between corresponding pixels across all spectral channels, thereby providing a quantitative measure of spectral reconstruction accuracy that may guide generator network training.

In embodiments, a spatial discriminator 420 may receive the high-resolution HSI image 418 and the modified RGB image 414 as inputs and may evaluate whether the spatial characteristics of the high-resolution HSI image 418 match those of the modified RGB image 414. For example, the spatial discriminator 420 may analyze spatial structure, edge sharpness, and/or texture patterns to determine whether the generated high-resolution hyperspectral image may exhibit spatial detail consistent with the RGB reference image, thereby providing adversarial feedback to encourage spatial fidelity.

In embodiments, a spatial loss function 422 may compare the high-resolution HSI image 418 with the modified RGB image 414 to evaluate spatial detail preservation. In embodiments, the spatial loss function 422 may comprise a perceptual loss function based on feature maps extracted from a pre-trained neural network. For example, the spatial loss may extract feature representations from intermediate layers of a VGG network for the generated hyperspectral image and/or the RGB reference, thereby computing the mean squared difference between these feature maps to evaluate perceptual similarity rather than pixel-level similarity.

In embodiments, the generator network 416 may be trained using a combined loss function that may incorporate feedback from the spectral loss function 438, the spatial loss function 422, the spectral discriminator 436, and/or the spatial discriminator 420. The adversarial training may optimize both spatial fidelity and spectral accuracy simultaneously, enabling the generator network 416 to produce high-resolution hyperspectral images that may preserve both the spatial detail of the RGB camera and the spectral information of the hyperspectral camera.

In embodiments, the spatial loss function 422, the spatial discriminator 420, the spectral loss function 438, and the spectral discriminator 436 may be in communication with the generator network 416 and may provide feedback signals that may guide iterative training and retraining of the generator network 416. During training, the generator network 416 may receive gradient signals computed from one or more of those four feedback sources. In such embodiments, the spatial loss function 422 may compute a gradient that may indicate how to adjust the generator's parameters to improve spatial similarity between the high-resolution HSI image 418 and the modified RGB image 414.

In embodiments, the spectral loss function 438 may compute a gradient that may indicate how to adjust the generator's parameters to improve spectral accuracy between the low-resolution HSI image 434 and the aligned HSI image 430. In embodiments, the spatial discriminator 420 may provide adversarial feedback that may encourage the generator network 416 to produce high-resolution hyperspectral images with spatial characteristics indistinguishable from real RGB images.

In embodiments, the spectral discriminator 436 may provide adversarial feedback that may encourage the generator network 416 to produce hyperspectral data with spectral characteristics indistinguishable from real hyperspectral measurements. The combined feedback from these four sources may enable the generator network 416 to learn a mapping that may preserve both spatial detail and spectral fidelity simultaneously. In embodiments, the feedback signals may be weighted and combined according to a loss weighting scheme that may balance the relative importance of spatial preservation, spectral accuracy, and adversarial realism. For example, the combined loss function may apply weighting coefficients such that the coefficients may be optimized through cross-validation to achieve optimal balance between spatial and spectral quality metrics.

In embodiments, the generator network 416 may be retrained periodically using new training data comprising additional paired RGB and hyperspectral images, with the feedback mechanisms continuing to refine the network's ability to generate high-quality super-resolution hyperspectral images across diverse retinal imaging conditions.

Thus, super-resolution network architecture 400 provides automated fusion of high-resolution spatial information with multi-channel spectral information through neural network-based processing that may overcome the fundamental resolution limitations of hyperspectral imaging hardware. The preprocessing workflow of FIG. 4A may eliminate illumination artifacts and may establish pixel-level spatial correspondence between RGB and hyperspectral modalities, enabling the super-resolution network of FIG. 4B to generate high-resolution hyperspectral images that may combine the spatial detail of RGB cameras with the spectral richness of hyperspectral cameras. In embodiments, the dual-discriminator adversarial training architecture may ensure that the generated images may preserve spatial fidelity and spectral accuracy simultaneously, producing image data with properties that may not be directly capturable by either camera alone. This approach may eliminate the need for sequential image acquisition and/or manual post-acquisition alignment present in known workflows, thereby providing simultaneous spatial and spectral information suitable for quantitative biomarker extraction and disease detection applications.

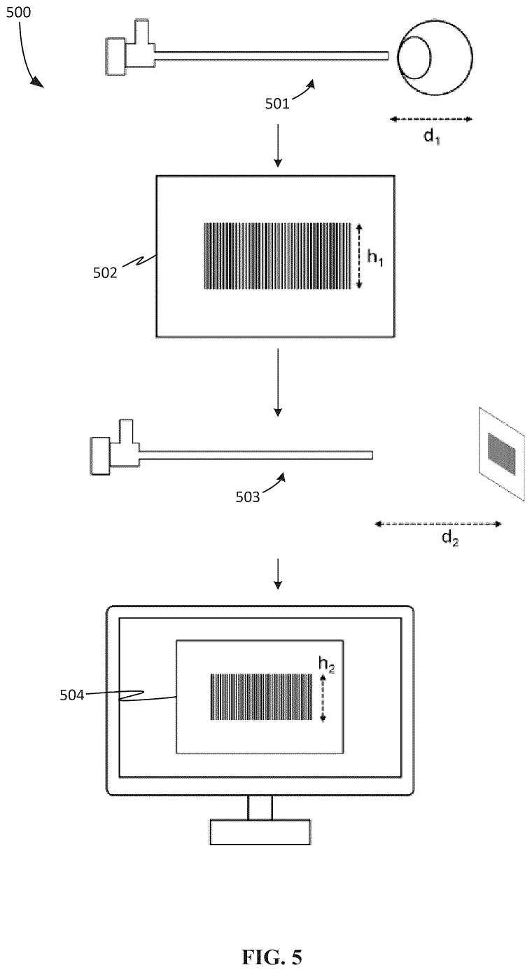

FIG. 5 shows a calibration workflow 500 in accordance with aspects of the present invention. In embodiments, an optical assembly (e.g., optical assembly 200 of FIG. 2) may be used to perform calibration measurements that may establish a conversion factor relating pixel dimensions in captured images to physical dimensions in retinal tissue. The calibration workflow 500 may enable accurate determination of vessel diameters and other morphological measurements from the high-resolution hyperspectral images generated by the super-resolution network architecture of FIG. 4B. The calibration workflow 500 may compensate for variations in optical magnification, imaging distance, and/or lens characteristics that may affect the relationship between pixel measurements and physical dimensions, thereby ensuring that quantitative biomarker measurements may be reported in standardized physical units suitable for clinical interpretation, longitudinal monitoring, and/or cross-study comparisons.

In embodiments, at operation 501, the optical assembly may be positioned at a first distance d₁ from the eye of a subject to capture retinal images. The endoscopic optical probe (e.g., endoscope 209 of FIG. 2) may be aligned with the cornea surface to image retinal blood vessels and tissue structures. The first imaging sensor (e.g., first imaging sensor 204 of FIG. 2) and the second imaging sensor (e.g., second imaging sensor 207 of FIG. 2) may simultaneously capture images through the beam splitter (e.g., beam splitter 201 of FIG. 2). In some embodiments, the distal tip of the endoscopic optical probe may be positioned proximate to or in contact with the cornea while a fixed focal length lens is adjusted until the retina is in focus, and the distance from the probe tip to a back of the retina may be treated as an object distance d₁ (optionally approximated using an eyeball diameter). A calibration target 502 may be displayed showing a pattern of known physical dimensions. In embodiments, the calibration target 502 may comprise vertical bars with a known height h₁ in micrometers unit. The captured image may record the calibration pattern, thereby enabling measurement of the pattern height in pixel units.

In embodiments, at operation 503, the optical assembly may be repositioned to a second distance d₂ from a physical calibration target to capture a reference image. The calibration target may comprise a printed pattern with known physical dimensions, such as vertical bars with a precisely measured height, as shown. In embodiments, while maintaining substantially the same focus setting used for retaining retinal imaging, the calibration target may be positioned near the distal tip of the endoscopic optical probe and moved along the optical axis until the calibration target is in focus, thereby defining an image distance d₂ corresponding to the calibrated focus configuration. The optical assembly may capture an image of this physical calibration target using the same optical configuration and sensor settings used for retinal imaging. A control computer (e.g., control computer 220 of FIG. 2 or a computer/system in communication with control computer 220) may measure the height of the calibration pattern in the captured image in pixel units.

In embodiments, a display 504 may present the captured calibration image showing the pattern with measured height h₂ in pixel units. The control computer 220 (or a computer/system in communication with control computer 220) may compute a magnification factor by comparing the known physical height of the calibration pattern with the measured pixel height in the captured image. For example, an eye magnification term may be computed as M_{eye}=d₂/d₁. A pixel-to-distance conversion may be computed as

m_{conversion}=d₂/d₁×h₁/h₂

where h₁ is a known physical height of a feature on the calibration target and h₂ is a measured pixel height of that feature in the captured image. Physical vessel diameters may be computed as:

Size in μm=Size in pixel×m_{conversion}.

In embodiments, the magnification factor may be calculated as the ratio of physical dimension to pixel dimension. The control computer may store the magnification factor for subsequent use in converting pixel-based measurements from retinal images to physical dimensions in micrometers.

In embodiments, the calibration workflow 500 may be performed periodically to account for changes in optical alignment, focus settings, and/or imaging distance. In embodiment, the calibration may ensure that vessel diameter measurements and other morphological measurements derived from the high-resolution hyperspectral images accurately reflect true physical dimensions in retinal tissue.

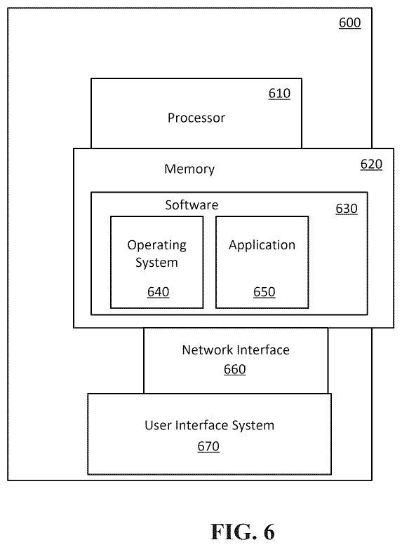

FIG. 6 shows a schematic diagram illustrating components of a computing device (e.g., control computer 220 of FIG. 2 and/or a computer/system in communication with control computer 220) in accordance with aspects of the present invention that may be used in certain implementations described herein. The computing device can be representative of a computing device on which a software analysis program is executed as well as the assemblies, methods, and workflows described herein with respect to FIGS. 2-5. Referring to FIG. 6, computing device 600, can represent a personal computer, a mobile device, a tablet, a laptop computer, a desktop computer, and/or a server, as some examples. Accordingly, more or fewer elements described with respect to computing device 600 may be incorporated to implement a particular computing device. For example, control computer 220 may include one or more processors/microprocessors and one or more communications interfaces; and a computer/system in communication with control computer 220 may be in the form of a personal computer, server, etc.

Referring to FIG. 6, computing device 600 can include at least one processor 610, a memory 620, software 630 that includes operating system 640 and application 650 stored in the memory 620, network interface 660, and user interface 670. At least one processor 610 processes data and performs operations according to instructions of software 630 (which may include instructions for performing the functionalities described with respect to optical assembly 200 and/or control computer 220 and/or computer/system in communication with control computer 220 as described throughout FIGS. 2-5). The instructions of application 650 may be loaded into computing device 600 and run on or in association with the operating system 640. Application 650 can include the assemblies, methods, and workflows described herein with respect to FIGS. 2-5. Memory 620 may comprise any computer readable storage media readable by at least one processor 610 and capable of storing software 630 including application 650. Memory 620 (and any computer readable storage media forming memory 620) does not consist of propagating signals nor is it considered transitory media.

Computing device 600 can further include a user interface 670, which may include input/output (I/O) devices and components that enable communication between a user and the computing device 600 such as, but not limited to, a display, keyboard, mouse, microphone, and speakers. Computing device 600 may also include a network interface 660 that allows the system to communicate with other computing devices, including server computing devices and other client devices, over a network. In embodiments, network interface 660 provides communication between computing device 600, imaging sensors (e.g., first imaging sensor 204 and/or second imaging sensor 207 of FIG. 2), and/or a database storing image data, to receive and/or obtain a first image having high spatial resolution and a second image having spectral information, where the first image and the second image are captured simultaneously using a common endoscopic optical probe. In embodiments, network interface 660 can include wired and/or wireless interfaces of one or more communication protocols and/or ports (e.g., for Wi-Fi or Ethernet, BLUETOOTH, near field communication (NFC), etc.).

The following provide example implementations and prototypes of a high-resolution hyperspectral imaging system as described herein.

In a high-resolution hyperspectral imaging system for the retina embodiment, the system may combine the speed of a snapshot hyperspectral camera with the spatial resolution of an RGB camera to provide enhanced spatial resolution for detection and monitoring of eye conditions. In embodiments, the system may be configured to measure oxygen saturation in the retina, a critical factor in diagnosing various ocular conditions. In embodiments, the system may be tested on a calibrated color chart, a model phantom of the eye, and on biological tissues to validate spatial resolution enhancement and spectral fidelity preservation.

In a high-resolution hyperspectral imaging system for the retina embodiment, a hardware system may be constructed by connecting an endoscope with a fixed focal length lens to provide magnification. Illumination may be provided through a fiber optic wire that travels in the same direction as the scope. An outer diameter of the scope may be 3 mm, which is comparable to the diameter of the pupil of the mice. The endoscope may be connected to a 10-90 beam splitter and diverts the light into an RGB camera and a hyperspectral snapshot camera. This unbalanced division of light may be chosen because the HSI camera may require significantly more light compared to the RGB camera due to the number of filters it has. Fixed-focal length lens may be used to connect the cameras to with the scopes. The RGB camera may use a 50 mm lens and the HSI camera may use a 75 mm lens. The snapshot camera may capture 16 bands of images at raster size of 270×512 pixels each band in the wavelengths 460-600 nm. Bandpass Fabry-Perot filters may be used and may be arranged in an alternating pattern. The RGB camera may be a scientific CMOS camera and can acquire images at raster size of up to 3840×3040 pixels. The illumination may be provided by a Xenon lamp through a flexible optic fiber to the endoscope. The entire system may be fixed in place vertically, with the scope pointing down toward the imaging subject.

Different types of scopes may be used for different applications, for example, a laparoscope and/or a small industrial borescope may be used. The laparoscope may be used in surgical settings. It has a length of 40.5 cm and a diameter of 0.4 inches (10 mm). The borescope may be used to image the retina of small animals, as well as small vascular structures. It has a length of 80 mm and an outside diameter of 1.65 mm. For the borescope, fixed-focal length lenses with focal lengths of 50 mm and 75 mm may be used for the RGB and the HSI cameras, respectively.

In the high-resolution hyperspectral imaging system for the retina embodiment, a software system to simultaneously capture hyperspectral image and RGB image at the same time may be used. Subjects were placed on top of a translating platform that can move up and down. The platform may be used to move the subjects into focus. Prior to imaging subjects, dark and white references may be acquired using a 95% reflective Spectralon target. The final image may be color-corrected using both the white and dark references using the following formula.

$$I = \frac{{I}_{raw} - {I}_{dark}}{{I}_{white} - {I}_{dark}}$$

where I_{raw} is the raw acquired image. I_{dark} is the dark reference, I_{white} is the white reference image, and I is the final corrected image. The raw image may be a mosaiced image, so a demosaicing method may be used to produce final hyperspectral images. The raster resolution of an RGB image may be 3840×3040×3 pixels, and the raster resolution of a hyperspectral image may be 512×270×16 pixels.

In the high-resolution hyperspectral imaging system for the retina embodiment, an automatic imaging workflow may be used. The workflow may have at least three stages: pre-processing, registration, and pan sharpening. For preprocessing, separate pipelines may be used for RGB images and hyperspectral images. The RGB image may be converted to grayscale. For the hyperspectral images, a 3×3 median filter may be applied to each individual channels before registration. The registration may be performed in the grayscale, high resolution images and the hyperspectral images using the SURF registration method. Registration may be performed in an image processing toolbox (e.g., MATLAB) with the assumption of similarity transformation (only rotation, translation, and scaling applied). The high-resolution image may be rescaled to a spatial dimension of 1020×540 pixels, which is twice the raster size of the hyperspectral image.

Image pansharpening may be performed using various methods, including: Brovey transformation, coupled-nonnegative matrix factorization, guided fusion PCA, Gram-Schmidt transformation, intensity-hue transformation, PanNet, and/or smoothing filter-based intensity modulation. In embodiments, these pansharpening methods may be implemented in Python. In this embodiment, the final image may have a spatial dimension of 1020×540×16 pixels, which is 4 times the raster size of the original hyperspectral image.

To ensure that the pan-sharpened, high resolution image preserves both the spectral fidelity (compared to the hyperspectral image captured by the snapshot camera) and the spatial fidelity (compared to the RGB image captured by the CMOS camera), at least two different metrics may be measured. To measure spectral fidelity, a root mean square error (RMSE) may be used. Accordingly, the pansharpened image may be down-sampled into the same dimension as the original, median filtered hyperspectral image. If i_{PAN} is the down-sampled pansharpened image, and i_{HSI} is the hyperspectral image. RMSE may be minimized when there is a similarity in the spectral profile:

$$R M S E = \sqrt{\frac{{\sum }_{all channels} {\sum }_{all pixels} {( {i}_{PAN} - {i}_{HSI} )}^{2}}{{\sum }_{all channels} {\sum }_{all pixels} 1}}$$

To measure spatial fidelity, a structurally similarity index measure (SSIM) may be used. SSIM relies on the measurements of the variances and the covariances between two images of similar sizes. If μₓ, σₓ, σ_{xy} are the mean and variance of image x, and the covariances between images x and y, respectively, then the formula for SSIM may be given as:

$$S S I M ( x , y ) = \frac{( 2 {μ}_{x} {μ}_{y} + {c}_{1} ) ( 2 {σ}_{xy} + {c}_{2} )}{( {μ}_{x}^{2} + {μ}_{y}^{2} + {c}_{1} ) ( {σ}_{x}^{2} + {σ}_{y}^{2} + {c}_{2} )}$$

In this formula, c1 and c2 are stabilizing variables. The SSIM for each channel may be compared with the high-resolution RGB images, then the SSIM scores may be averaged with equal weights given to every channel.

The pansharpened images resolve retinal vessels with higher details that are not seen in the hyperspectral image. By measuring the physical distances between known points in the phantom retina, the spatial resolution of the original hyperspectral image may be calculated to be around 0.2 mm/pixel, whereas the spatial resolution of the pansharpened image to be around 0.1 mm/pixel. The mean spectra in the region of interests (ROI) may also be compared. This ROI contains a section of the blood vessels, such that the pansharpened image can potentially be used to estimate different biosignature of the retinal blood vessels.

In the high-resolution hyperspectral imaging system for the retina embodiment, the system combines the speed of a snapshot hyperspectral camera with the spatial resolution of an RGB camera. The enhanced spatial resolution provided by the hybrid imaging system is important for the detection and monitoring of eye conditions such as diabetic retinopathy and early signs of Alzheimer's disease. Furthermore, the hyperspectral imaging system could measure oxygen saturation in the retina, a critical factor in diagnosing various ocular conditions. This system represents a significant advancement in retinal imaging technology, offering real-time acquisition of high-resolution hyperspectral images.

In a dual-camera high-resolution hyperspectral imaging system for the retina embodiment, the system may be configured to integrate hyperspectral imaging with high-resolution RGB imaging to capture detailed retinal images with enhanced spatial resolution. In embodiments, the system may employ pan-sharpening algorithms to enhance the spatial resolution of hyperspectral images, thereby combining the spectral detail of hyperspectral imaging with the spatial clarity of RGB imaging. In embodiments, the system may develop algorithms to estimate the diameter of retinal vessels and the oxygenation rate from the fused image data. In embodiments, the system may be validated by imaging a mouse retinal phantom and by imaging mice under anesthesia to demonstrate the ability to resolve fine structural details, including small blood vessels.

In the dual-camera high-resolution hyperspectral imaging system for the retina embodiment, an acquisition system (e.g., a hyperspectral retinal imaging system) may include a snapshot HSI camera, a high-resolution RGB imaging camera, a 70:30 optical beam splitter, imaging endoscopes, and fixed focal length imaging lenses. In such embodiments, the HSI snapshot camera may capture 16 bands of images at image resolution of 270×512 pixels from 460-600 nm. The RGB camera may be a CMOS camera and may be configured to acquire RGB images at pixel resolution of up to 2048×3072 pixels. The acquisition system may be designed for mouse retinal imaging and/or human retinal imaging by the use of a larger endoscope. Illumination may be provided from a Xenon illumination source through a fiber optic wire that travels in the same direction as the scope. Finally, a series of fixed focal length lenses may be used to provide magnification of the images.

Prior to imaging an animal retina, the acquisition system may be validated on a series of mouse retinal phantoms. In such instances, a mouse retina phantom may be 3-D printed. A lens holder may be used to mimic the natural curvature and dimensions of a mouse lens. For the mouse lens, a semi-spherical lens with a diameter of 3.10 mm and a focal length of 3 mm may be used. The retina phantom may be used to validate a determined magnification factor formula.

High-Resolution Images may be produced by creating an automatic imaging workflow. The workflow may have three stages: pre-processing, registration, and pan-sharpening. For preprocessing, separate pipelines may be used for RGB images and hyperspectral images. The RGB image may be converted to grayscale. The grayscale, high resolution images may be registered under the conditions that only translation, scaling, and rotation are involved (similarity transformations). Using manual examination, the rotation and translation necessary to produce proper registration may be determined.

In embodiments, the foregoing pan-sharpened images may be produced using one or more of the following methods: Bicubic interpolation, Brovey algorithm, Generalized intensity-hue-saturation, Adaptive Gram-Schmidt, Gram-Schmidt, High-pass filtering using 13×13 box filter, Undecimated/“à trous” wavelet substitution, Convolutional neural network, Generative adversarial network, Unsupervised generative adversarial network, and/or U-Net.

In the dual-camera high-resolution hyperspectral imaging system for the retina embodiment, observed system optical performances show that the nearest imaging distance of the system was 1.9 mm. Experiments have shown that the imaging distance of the mouse retina may range from 3.5-4 mm, which means that the system is fully capable of imaging mouse retina in focus. Specifically, the test results show that the HSI camera can achieve spectral accuracy comparable to that of a spectrometer. A measured magnification factor of the mouse eye may be 1.2×, the optical magnification of the endoscope system at imaging distance to be 2.15 μm/pixel, and the conversion factor for retinal image to be 1.80 μm/pixel. Comparing the lengths of strands calculated against the actual length gave an error rate of 7.6±5.6%. This shows that a conversion algorithm produces reasonable approximation of small details within the phantom retina.

In short, the dual-camera high-resolution hyperspectral imaging system for the retina embodiment, comprises a dual-camera system combining hyperspectral imaging (HSI) and high-resolution RGB imaging to capture detailed retinal images. The system produces high-resolution images of animal retinal phantoms, resolving fine structural details crucial for analysis. By applying pan-sharpening algorithms, the spatial resolution of hyperspectral images is enhanced, thereby merging the spectral detail of HSI with the spatial clarity of RGB imaging. Imaging results confirm the embodiment's ability to detect small blood vessels, essential for identifying subtle retinal changes and abnormalities. Furthermore, deep learning-based approaches, such as convolutional neural networks (CNNs) and generative adversarial networks (GANs), deliver higher fidelity in both spatial and spectral dimensions.

This embodiment may be configured to extract and validate biomarkers from the pansharpened images, focusing on vessel diameter and oxygenation rate. These biomarkers reveal important details about retinal metabolic activity and demonstrate the strengths of the system. High-resolution RGB images captured small structures with minimal motion blur, while hyperspectral images provided the necessary spectral data. Notably, RGB images mitigated the motion blur introduced by the hyperspectral camera's longer exposure time, improving overall image quality. hyperspectral imaging.

In a retina phantom model for hyperspectral imaging embodiment, phantom designs for lens holders may be used. For example, a human retina phantom may have a pupil diameter of 7 mm. The retina structure may comprise blood vessels, optic disc, and/or a simulated foreign body. A rat retina phantom may have an iris diameter of 6 mm and focal length of 6 mm. Because mouse and rat eyes differ in size by linear factor of 1.9-2.0, a mouse phantom opening models an interior diameter of 3.1 mm (~0.12 in) with an opening of 2.7 mm (~0.11 in). In such instances, a lens holder may be created to model the lens size, axial length, and/or focal length of a mouse eye. The lens holder may accommodate a model mouse lens with a diameter of 4 mm (~0.16 in) and a height of 2.3 mm (~0.09 in), effectively simulating the natural curvature of a mouse lens. The lens may be made of borosilicate glass material, with a refractive index of 1.5168. Similarly, healthy adult C57BL6 mice lenses may have a maximum refractive index of ~1.55. In mice, the lens occupies 65-75% of the eye, so the anterior chamber phantom may use a depth (focal length) of 1.5 mm (~0.059 in). The design process may be performed using computer-aided design (CAD) software. The design may use white filament as its material, which has an average assumed rate of shrinkage range of 0.8% to 3%. The model may be 3D printed using a 0.4 mm (about 0.02 in) nozzle print head.

In the retina phantom model for hyperspectral imaging embodiment, the blood vessels (arteries and veins) and the vitreous humor may also be modelled. To simulate the blood vessels, carbon fibers with diameter of about 5 μm may be used. To simulate vitreous humor and mimic its consistency, lubricant eye drops may be used to fill the inner portion of the model.

The mouse retinal phantom may be used to estimate the size of blood vessels and to determine a magnification factor of a mouse retina imaging system. The phantom model may be imaged with an RGB camera at a focal depth simulating that used in live retinal imaging setups. Software may be utilized to extract pixel length measurements of individual carbon fiber strands. A 1 mm marker with 0.1 mm increments may be imaged to calculate the magnification factor. The mouse eye phantom may be imaged using the same setup under two configurations: with the endoscope only and with the endoscope combined with the model lens. The measured pixel lengths of the carbon fibers under each configuration may be converted to millimeters using the corresponding magnification factor.

In the retina phantom model for hyperspectral imaging embodiment, blood vessels may also be modelled. For example, a phantom using gelatin may be built to simulate small blood vessels and validate the spectral ability of the HSI camera. Defibrinated sheep blood with 36% hematocrit may be used to produce blood samples. The blood may be oxygenated by stirring it using a magnetic stirrer, the oxygenation rate may be confirmed using a spectrophotometer.

Sodium dithionite solution may be prepared by mixing 1 gram of sodium dithionite (Na₂SO₄) salt into 40 mL of phosphate buffer saline (PBS) solution at room temperature and 7.4 pH. The PBS solution may be sparged with nitrogen gas beforehand to remove dissolved oxygen. The sodium dithionite-PBS solution may be diluted into blood to produce concentrations of sodium dithionite at 0.5, 1.0, 1.4, 2.5, and 4.0 mg/mL of blood. The gelatin body may be made using a combination of 10% gelatin to 90% deionized water. Using hematocrit tubes with inner diameters of 1.1 mm, blood samples of different sodium dithionite concentrations may be drawn.

In the retina phantom model for hyperspectral imaging embodiment, a modified topical fundus endoscopic imaging (TEFI) system may be utilized for dual-camera imaging, integrating both hyperspectral (HSI) and RGB modalities. The system may include a snapshot HSI camera, a high-resolution RGB camera, a 70:30 optical beam splitter, imaging endoscopes, and fixed focal length lenses. The HSI camera may capture 16 spectral bands with an image resolution of 270×512 pixels within the 610-850 nm range. The RGB camera, a scientific CMOS model, may capture images at resolutions up to 2048×3072 pixels. Both systems may be mounted on a translation stage, positioned atop a rotational platform. This configuration provides the system with four degrees of freedom (three rotational and one translational) for targeting various objects. A separate translating stage may be used to allow for vertical adjustment of the subject to bring the pupil into focus. Exposure times may be set at 100 ms for the RGB camera and 150 ms for the HSI camera. A halogen light source with a spectral range extending into the near-infrared (NIR) may be used to provide illumination. The acquisition of gelatin phantoms may be performed using a similar setup. The snapshot camera may be connected to a surgical laparoscope with a 10 mm diameter tip placed 10 mm from the blood sample surface. Halogen light may be used as the illumination source.

In the retina phantom model for hyperspectral imaging embodiment, dark and white references may be acquired using a 95% reflective Spectralon target. The raw image may be normalized using white and dark reference. To analyze the retinal phantom images, pixels belonging to the vessels and pixels belonging to the background may be selected and used to compare their spectral differences.

Best-fit theoretical curves may be used to produce oxygenation curves at 100% and 0% to analyze the gelatin phantom. All curves may be normalized by their isosbestic value at 805 nm. Assuming that oxygenated and deoxygenated hemoglobin are the two primary chromophores, then the resulting spectra may be simply the linear combination of the spectra of oxygenated and deoxygenated hemoglobin. If the theoretical reflectance of fully oxygenated blood is Roxy, the reflectance of fully deoxygenated blood is R_{deoxy}, the reflectance of our sample is Rₛₐₘₚₗₑ, then we estimated the oxygenation rate using the formula:

$$S {O}_{2} \% of sample = \frac{{R}_{sample} - {R}_{deoxy}}{{R}_{oxy} - {R}_{deoxy}} \times 100$$

The equation should work for any wavelength. Since there are considerable differences in normalized reflectance between oxygenated and deoxygenated hemoglobin at 650-700 nm, oxygenation rates may be calculated by using the summed spectra from 650-700 nm.

In the retina phantom model for hyperspectral imaging embodiment, a mouse retina model may be designed to mimic the optical characteristics of mice. The phantom body may be 3D printed. A gelatin phantom may be developed that mimics the spectral characteristics of veins and arteries. Blood samples may be produced using sodium dithionite to generate predictable oxygen saturation levels. This design includes more accurate anatomical modeling, selecting an optical medium that mimics vitreous humor, and a validation study using a gelatin phantom. The development of such phantoms may be used to test the spatial and spectral abilities of HSI cameras and can be translated into animal studies. This approach provides a simple and effective noninvasive calculation of retinal blood vessel diameters. This phantom serves as a versatile tool for standardizing imaging protocols, developing new imaging technologies, and reducing reliance on live animal models. By providing a reproducible, customizable platform, this work advances retinal research, with potential applications in surgical training, educational settings, and/or preclinical testing of retinal therapies.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts that would be recognized by one skilled in the art are intended to be within the scope of the claims.

What is claimed is:

1. A system for imaging a retina, comprising:
an optical assembly comprising an endoscopic optical probe, the optical assembly being configured to simultaneously direct light from the retina to a first imaging sensor and a second imaging sensor,
wherein the first imaging sensor is configured to capture a first image having higher spatial resolution,
wherein the second imaging sensor is configured to capture a second image having spectral information across multiple wavelengths,
wherein simultaneously directing light from the retina to the first imaging sensor and the second imaging sensor comprises directing the light through a beam splitter, and
wherein the beam splitter is configured to divide light from a single optical path into separate optical paths toward the first imaging sensor and the second imaging sensor.

2. The system of claim 1, wherein the first imaging sensor and the second imaging sensor are optically coupled to a common endoscopic optical probe.

3. The system of claim 1, wherein the first imaging sensor and the second imaging sensor are arranged to image a same field of view of the retina.

4. The system of claim 1, wherein the first image and the second image are captured during a single image acquisition event.

5. The system of claim 1, wherein the first imaging sensor comprises a high-resolution visible-light camera.

6. The system of claim 1, wherein the second imaging sensor comprises a snapshot hyperspectral imaging camera.

7. A computer program product comprising:
one or more computer-readable storage media; and
program instructions stored on the one or more computer-readable storage media to perform operations comprising:
receiving a first image having high spatial resolution and a second image having spectral information, wherein the first image and the second image are captured simultaneously using a common endoscopic optical probe;
generating a high-resolution hyperspectral image by combining spatial information from the first image with spectral information from the second image;
identifying pixels corresponding to retinal blood vessels within the high-resolution hyperspectral image;
determining oxygenation values for the identified blood vessel pixels using the spectral information; and
determining physical dimensions of the retinal blood vessels using spatial measurements derived from the high-resolution hyperspectral image.

8. The computer program product of claim 7, wherein the operations further comprise spatially aligning the first image and the second image.

9. The computer program product of claim 7, wherein generating the high-resolution hyperspectral image comprises applying a trained neural network.

10. The computer program product of claim 7, wherein the first image and the second image are captured during a single image acquisition event.

11. The computer program product of claim 7, wherein the operations further comprise performing radiometric calibration on at least one of the first image and the second image using a white reference image.

12. The computer program product of claim 7, wherein determining the oxygenation values comprises converting white-reference corrected intensities to optical density values and performing multi-wavelength curve fitting to a hemoglobin absorption model to estimate oxygen saturation.

13. The computer program product of claim 7, wherein identifying pixels corresponding to retinal blood vessels comprises applying a trained segmentation neural network to the high-resolution hyperspectral image.

14. The computer program product of claim 7, wherein the operations further comprise generating, using a trained segmentation network, a segmentation mask of at least one retinal structure comprising a macula and an optic nerve head.

15. A method for imaging a retina, comprising:
simultaneously directing light from the retina to a first imaging sensor and a second imaging sensor;
capturing, by the first imaging sensor, a first image having higher spatial resolution;
capturing, by the second imaging sensor, a second image having spectral information;
generating a high-resolution hyperspectral image by combining spatial information from the first image with spectral information from the second image;
identifying pixels corresponding to retinal blood vessels within the high-resolution hyperspectral image;
determining oxygenation values for the identified blood vessel pixels using the spectral information; and
determining physical dimensions of the retinal blood vessels using spatial measurements derived from the high-resolution hyperspectral image.

16. The method of claim 15, further comprising spatially aligning the first image and the second image.

17. The method of claim 15, wherein the first imaging sensor comprises a high-resolution visible-light camera, and
wherein the second imaging sensor comprises a snapshot hyperspectral imaging camera.

18. The method of claim 15, wherein simultaneously directing light from the retina to the first imaging sensor and the second imaging sensor comprises directing the light through a beam splitter, wherein the beam splitter is configured to divide light from a single optical path into separate optical paths toward the first imaging sensor and the second imaging sensor.

19. The method of claim 15, wherein the first imaging sensor and the second imaging sensor are optically coupled to a common endoscopic optical probe, and
wherein the first imaging sensor and the second imaging sensor are arranged to image a same field of view of the retina.

20. The method of claim 15, wherein the first image and the second image are captured during a single image acquisition event.
